(19) United States
(12) Patent Application Publication
(10) Pub. No.: US 2024/0353395 A1
(43) Pub. Date: Oct. 24, 2024

(54) METHOD AND SYSTEM FOR MEASURING CELL-MEDIATED IMMUNITY TO ADENO-ASSOCIATED-VIRUS INFECTION

(71) Applicant: Plexision, Pittsburgh, PA (US)

(72) Inventors: Rakesh Sindhi, Pittsburgh, PA (US); Chethan Ashokkumar, Pittsburgh, PA (US)

(21) Appl. No.: 18/640,027

(22) Filed: Apr. 19, 2024

Related U.S. Application Data
(60) Provisional application No. 63/497,002, filed on Apr. 19, 2023.

(51) Int. Cl. G01N 33/50

(52) U.S. Cl. CPC G01N 33/5047; G01N 2333/015; G01N 2333/165; G01N 2333/70575; G01N 2800/50; G01N 2800/52

(57) ABSTRACT

A method for measuring cell-mediated immunity to AAV-related gene therapy is provided. The method includes measuring cell-mediated immunity to adeno-associated viruses (AAV) and the transgene carried by these viruses which are being used for gene replacement therapy to treat genetic disorders. These measurements are used to a) estimate the risk of failure, b) the adequacy of immunosuppression, c) adjustment of immunosuppression d) to achieve desired suppression of the immune response, and e) to determine whether a patient about to undergo gene therapy is likely to develop and immune response to the therapy or not. The related embodiment which measures several complement or related proteins on cells is aimed at measuring the risk of TMA, its severity and its response to treatment. The present invention also provides a kit for measuring cell mediated immunity to AAV gene therapy.

### CROSS REFERENCE TO RELATED APPLICATION(S)

This application claims the priority to incorporates by reference the entire disclosure of U.S. provisional patent application No. 63/497,002 filed on Apr. 19, 2023 titled “METHOD AND KIT FOR MEASURING CELL-MEDIATED IMMUNITY TO ADENO ASSOCIATED VIRUS AND ASSOCIATED GENE REPLACEMENT PRODUCTS USED FOR GENE THERAPY”.

### FIELD OF INVENTION

Embodiments of the present invention relates to immunological studies and more particularly it relates to a method and a system for measuring cell-mediated immunity to adeno-associated-virus or AAV in the course of its use for gene therapy.

### BACKGROUND

Recently, gene therapy is being used to cure many genetic disorders. Wang et al., 2019, in their paper, disclosed that the gene therapy uses natural or synthetic AAV particles as vehicles to carry gene replacement product or transgenes. The combination is injected into a person with a defective gene. Most such therapy is performed with recombinant AAV (rAAV). In the rAAV, most viral genome is replaced by a gene expression cassette which carries the gene replacement product or transgene.

Engineered viruses are being used increasingly in gene therapy. These viruses include but are not limited to adenovirus, adeno-associated virus, lentivirus, herpes simplex virus, etc.

Several different types of AAVs exist in nature. The different AAVs are structurally similar. Calcedo et al., 2009, in their paper, disclosed that exposure to these viruses is common and leads to the development of neutralizing antibodies in 10 to 40% of the population. Antibodies to one type of AAV can cross-react with another AAV and vice versa.

The success of gene therapy depends on avoiding an immune response directed toward the AAVs and the gene replacement product or transgene. Cell-mediated immunity is a second component of the immune response. Cells that mediate this immune response include but are not limited to circulating T-cells, B-cells, natural killer cells, monocytes and dendritic cells. This immune response can lead to fever, altered blood chemistry tests such as elevated liver function tests, damage to other organ systems such as pneumonia. An unchecked antibody or cellular immune response can cause failure of gene therapy.

The immune response to the gene therapy is managed with immunosuppressants which have been used to prevent rejection after solid organ transplantation. These immunosuppressants include but are not limited to calcineurin inhibitors such as tacrolimus, antiproliferative agents such as rapamycin, mycophenolate mofetil, steroids and antibodies which deplete or suppress the function of T-cells and B-cells and other immune cells. These immune cells make up the population of circulating white blood cells.

In some patients, gene therapy causes other side effects such as thrombotic microangiopathy (TMA). This condition can occur after gene therapy and causes haemolytic anaemia due to breakdown of red blood cells; low platelet counts and damage to any number of organs, especially kidneys. Activation of coagulation and complement proteins is a common event in TMA. These proteins are expressed on the surface of many immune cells like platelets and monocytes.

Thus, during gene therapy, cellular immune responses to a particular AAV may measure the magnitude of the immune response and the effect of immunosuppression on this immune response. These measurements may be used to enhance the management of immunosuppression after gene therapy.

Hence, there is a need for a method and a kit for measuring cell-mediated immunity to AAV gene therapy.

### SUMMARY

In accordance with an embodiment of the present invention, a method for measuring cell-mediated immunity for viral antigens comprising adeno-associated-virus (AAV) viral particles and the gene replacement product is provided. The method includes mixing white blood cells of one or more subjects undergoing gene therapy with the AAV as a vehicle, virus particles, viral protein or peptides representing viral proteins or any particle representative of a gene replacement product or the transgene. The method also includes measuring a response using an inflammatory marker CD154 with or without CD40 which are expressed on stimulated white blood cells of one type or the other. These types include but are not limited to T-cells, B-cells, monocytes, natural killer cells or platelets.

In accordance with another embodiment of the present invention, the method for measuring cell-mediated immunity to AAV and the gene replacement product further includes generating a risk score using a computing device configured with a multivariate predictive algorithm based on immune cell responses and a plurality of variables associated with a subject. The method also includes determining risk of cell-mediated immunity, high or low, present or not, by a magnitude of a risk score. Thereby, the method enables measuring the risk of the cell-mediated immune response to the virus.

In accordance with yet another embodiment, the present invention also provides for a computing device configured to receive data representative of the response generated using a marker CD154 with or without CD40 expressed on the stimulated one or more white blood cells, run a multivariate predictive algorithm, and compute a risk score for the individual undergoing testing.

In accordance with an embodiment of the present invention, a kit for measuring cell-mediated immunity to AAV and the gene replacement product is provided. The kit includes reference antigen to bind with the one or more white blood cells of individuals to be tested for cell-mediated immunity. The kit enables rapid measurement of the risk of cell-mediated immunity, severity of immunity and whether this immunity has been reduced by the use of immunosuppression.

In accordance with another embodiment of the present invention, additional markers including but not limited to CD38, CD137, OX40, OX40 ligand, CD80, CD86, CD28, or CD69, cytokines such as IFNγ, TNF-α and IL-2, exhaustion markers such as PD-1 and PD-L1, apoptosis markers which measure activation of apoptotic enzymes such as caspases and cathepsins, and suppressive markers such as CD39, CD73, FOXP3, TGFB1 and IL-10 can be added to CD154 to measure the response of the one or more white blood cells to stimulation with viral antigens described in above mentioned embodiments.

In accordance with another embodiment of the present invention, additional markers including but not limited to the coagulation proteins like von Willebrand factor, and complement proteins C1q, C1s, C1R, CFB, C3, DGKE, CFH, CFI, CD46, CD55, and CD59, can be added to CD154 to measure the response of the one or more white blood cells to stimulation with viral antigens described in abovementioned embodiments. Increased expression of these proteins can be used to predict the likelihood of TMA.

In accordance with another embodiment of the present invention, the test or method can be used as a companion diagnostic for gene therapy which uses a specific AAV as a vehicle and the gene replacement product it carries. The purpose of this companion diagnostic includes but is not limited to determining whether the gene therapy is being rejected by the immune system, determining whether the cell-mediated immune response is being suppressed by immunosuppression, and identifying those individuals with a discordant antibody-mediated and the cell-mediated immune response. This discord may include those who have antibodies (seropositive) but not cell-mediated immunity or those who do not have antibodies (seronegative) but have cell-mediated immunity. The absence of cell-mediated immunity in seropositive individuals can permit successful use of the gene therapy with the use of some types of immunosuppression. Similarly, seronegative individuals with cell-mediated may undergo successful gene therapy. The presence of both types of immune response may lead to failure of the gene therapy or the need for stronger immunosuppression to suppress both types of immune responses. Thus, cell-mediated measurements combined with available tests of serologic status may be used to select patients and the type of immunosuppression for the gene therapy.

Cellular expression of coagulation or complement proteins can be used to predict the risk of TMA.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF ACCOMPANYING DRAWINGS

The disclosure will be described and explained with additional specificity and detail with the accompanying figures in which:

FIG. 1 illustrates an exemplary block diagram representation of a network architecture implementing a system for measuring cell-mediated immunity for viral antigens, in accordance with an embodiment of the present disclosure;

FIG. 2 illustrates an exemplary block diagram representation of a computer-implemented system, such as those shown in FIG. 1, capable of determining a risk associated with cell-mediated immunity to gene replacement therapy based on a magnitude of the risk score, in accordance with an embodiment of the present disclosure; and

FIG. 3 is a flow diagram representing steps involved in a method for measuring cell-mediated immunity for viral antigens, in accordance with an embodiment of the present invention.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

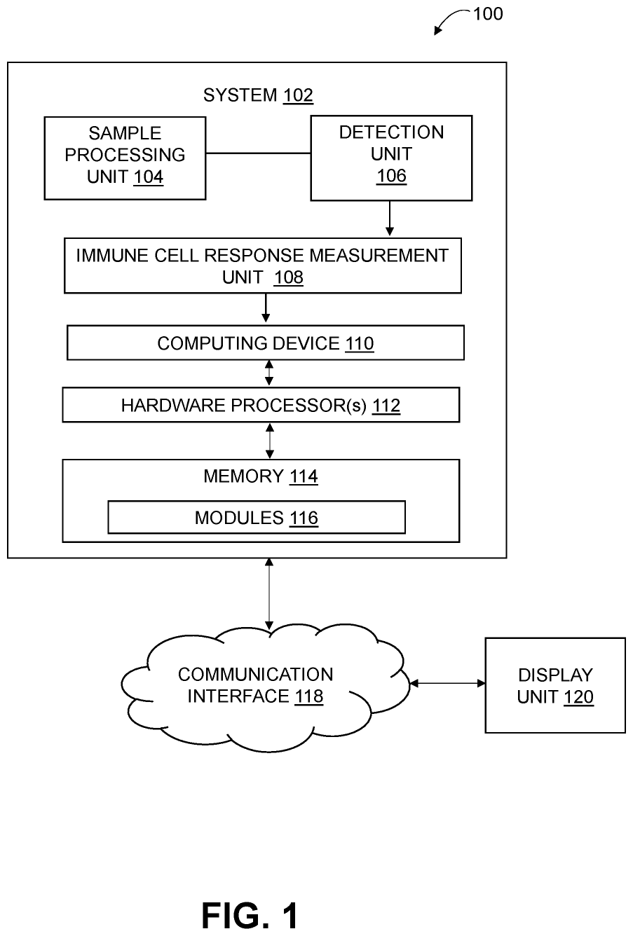

FIG. 1 illustrates an exemplary block diagram representation of a network architecture 100 implementing a system for measuring cell-mediated immunity for viral antigens, in accordance with an embodiment of the present disclosure.

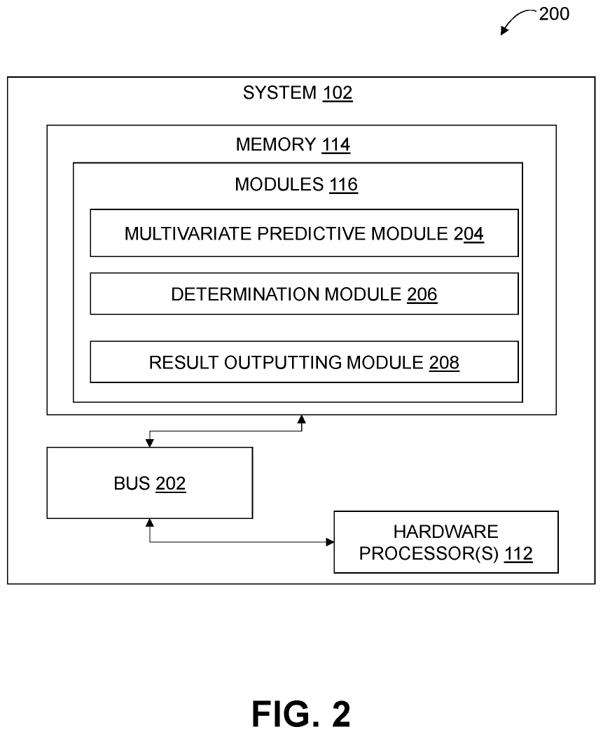

FIG. 2 illustrates an exemplary block diagram representation 200 of a computer-implemented system, such as those shown in FIG. 1, capable of determining a risk associated with cell-mediated immunity to gene replacement therapy based on a magnitude of the risk score, in accordance with an embodiment of the present disclosure.

In an embodiment of the present invention, a system 102 for measuring cell-mediated immunity for viral antigens is provided. The system 102 comprises a sample processing unit 104 configured to mix white blood cells of one or more subjects with viral antigens, a detection unit 106 configured to detect cell-bound markers indicative of immune activation with the viral antigens, an immune cell response measurement unit 108 configured to measure an immune cell response using markers comprising CD154 expressed on stimulated white blood cells, a computing device 110 comprises one or more hardware processors 112 configured to run a plurality of modules 116, and a memory 114 coupled to the one or more hardware processors 112. The memory 114 comprises the plurality of modules 116 in form of programmable instructions executable by the one or more hardware processors 112. The plurality of modules 116 comprises a multivariate predictive module 204 configured to generate a risk score based on a measured immune cell response and a plurality of variables associated with a subject, a determination module 206 configured to determine a risk associated with cell-mediated immunity to gene replacement therapy based on a magnitude of the risk score, and a result outputting module 208 configured to output, through a display communicatively connected to the one or more hardware processors, for visual presentation of data and results, based on a result of the magnitude of the risk score. Further, the system comprises a transceiver configured for wireless communication with one or more external devices, a bus 202 configured for facilitating data transfer between internal components, a communication interface 118 configured for connecting to one or more external networks, a display unit 120 configured for visual presentation of data and results, an input device configured for user interaction and data entry. The viral antigens comprise virus particles, viral proteins, and peptides representing viral proteins. The viral antigens comprise adeno-associated viruses (AAV) and transgenes carried by the adeno-associated viruses (AAV) for the gene replacement therapy. The detection of the cell-bound markers is performed using a detection method capable of detecting one of cell-bound and cell-derived markers from one of flow cytometry, mass cytometry, and single-cell RNA sequencing. The white blood cells comprise at least one of T-cells, T-helper cells (CD4), T-cytotoxic cells (CD8) and memory (CD45RO+) and naïve subsets (CD45RO−) of the T-cytotoxic cells, B-cells (CD19 or CD20), natural killer cells (CD16 and CD56) and monocytes (CD14).

In another embodiment of the present invention, the system further comprising measuring additional markers on a cell surface, wherein the additional markers comprise at least one of CD38, CD137, CD69, cytokines (IFNγ, TNF-α, IL-2), exhaustion markers (PD-1, PD-L1), and suppressive markers (CD39, CD73, FOXP3, TGFb1, IL-10). The plurality of variables associated with the subject comprises at least one of age, gender, ethnicity, time from diagnosis of infection with molecular studies, and use of immunosuppressive drugs. The magnitude of the risk score corresponds to a severity of an infection. The severity of infection is categorized into one of mild, moderate, and severe based on the magnitude of the risk score. The magnitude of the risk score also determines whether the response to vaccination results in decreased risk of infection or not.

In an embodiment of the present invention, a computing device 110 is provided. The computing device 110 is configured to receive data representative of the response generated using a marker CD154 expressed on stimulated white blood cells, run a multivariate predictive module 204, and compute a risk score for the individual undergoing testing. The computing device is configured to compute a risk score for the individual undergoing testing.

The processor corresponds to any type of computational circuit, such as but not limited to a microprocessor, a microcontroller, a complex instruction set computing microprocessor, a reduced instruction set computing microprocessor, a very long instruction word microprocessor, an explicitly parallel instruction computing microprocessor, a graphics processor, a digital signal processor or any other type of processing circuit. The processor may also include embedded controllers, such as generic or programmable logic devices or arrays, application specific integrated circuits, single-chip computers, smart cards, and the like.

The hardware processor may include, for example, microprocessors, microcomputers, microcontrollers, digital signal processors, central processing units, state machines, logic circuits, and/or any devices that manipulate data or signals based on operational instructions. Among other capabilities, hardware processor may fetch and execute computer-readable instructions in a memory operationally coupled with the system for performing tasks such as data processing, input/output processing, and/or any other functions. Any reference to a task in the present disclosure may refer to an operation being or that may be performed on data.

Though few components and subsystems are disclosed herein, there may be additional components and subsystems which is not shown, such as, but not limited to, assets, machinery, instruments, facility equipment, life safety devices, intensive care devices, treatment devices, emergency management devices, health care devices, laboratory devices, testing kits, any other devices, and combination thereof. The person skilled in the art should not be limiting the components/subsystems disclosed herein.

Those of ordinary skilled in the art will appreciate that the hardware disclosed may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

The memory may be volatile memory and non-volatile memory. The memory includes a risk score generation module according to the embodiments of the present invention. For example, the risk score generation module is configured to run the multivariate predictive algorithm for received data, where the data being representative of the response generated using a marker CD154 expressed on the stimulated one or more white blood cells. Further, the risk score generation module configured to generate the risk score and a report regarding level of risk of cell-mediated immunity to gene therapy based on a magnitude of the risk score for the individual.

A variety of computer-readable storage media may be stored in and accessed from memory elements. The memory elements may include any suitable memory device(s) for storing data and machine-readable instructions such as read only memory, random access memory, erasable programmable read only memory, electrically erasable programmable read only memory, hard drive, removable media drive for handling memory cards, Memory Sticks™, and the like.

In one embodiment, the computing device may be implemented in conjunction with modules including functions, procedures, data structures, and application programs for performing tasks or defining abstract data types or low-level hardware contexts. The risk score generation module may store in the form of machine-readable instructions on any of the abovementioned storage media and executed by the processor.

The components such as the transceiver, a communication interfaces, the display unit, the input device, and the cursor control are well known to the person skilled in the art and hence the explanation is thereof omitted.

In another embodiment of the present invention, a method for measuring cell-mediated immunity to viral particles and gene replacement products or transgene used in gene therapy is provided. The invention mainly focuses on measuring a risk of cell-mediated immunity, severity of this immunity, and assessing response to therapeutic interventions such as immunosuppressants or related medications.

In an embodiment, the method for measuring cell-mediated immunity to gene therapy products (viral particles or the gene replacement product) is provided. The method includes measuring cell-mediated immunity to adeno-associated viruses (AAV) and the transgene carried by these viruses which are being used for gene replacement therapy to treat genetic disorders.

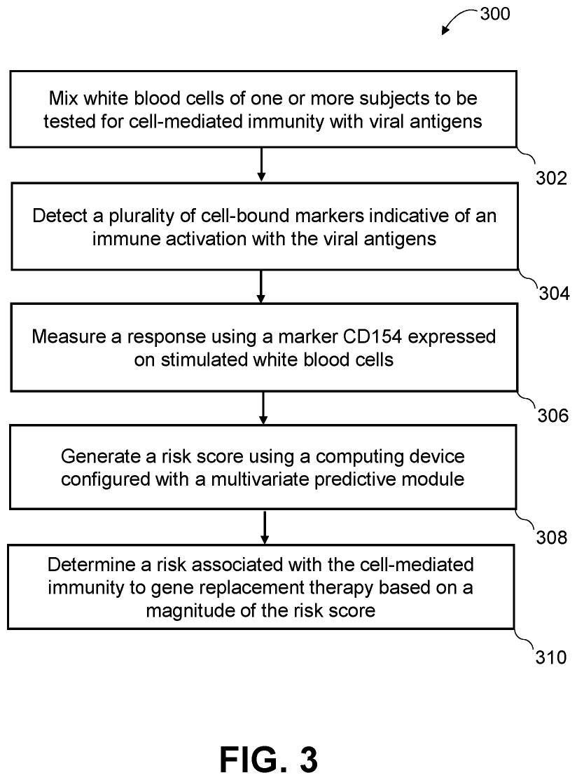

FIG. 3 is a flow diagram 300 representing steps involved in the method for measuring cell-mediated immunity for viral antigens, in accordance with an embodiment of the present invention. The method mainly includes testing one or more white blood cells of individuals undergoing, or about to undergo or those who have undergone gene therapy.

At step 302, the method for measuring cell-mediated immunity for viral antigens begins with mixing white blood cells of one or more subjects to be tested for cell-mediated immunity with viral antigen in the form of virus particles, viral protein, and peptides which represent the various viral proteins. The virus particles, viral proteins, and peptides representing viral proteins being known as viral antigens. The proteins present in the gene therapy product include but are not limited to viral protein, fragments of these proteins, and peptide mixtures representing the length of these various proteins and fragments. In one embodiment, the peptides which represent the various viral proteins are synthetic. In another embodiment, the viral antigens comprise adeno-associated viruses (AAV) and transgenes carried by the adeno-associated viruses (AAV) for gene replacement therapy.

In an alternative embodiment, the viral antigen or reference antigen is labeled with detectors comprising at least one of dyes, fluorescent dyes, and metallic labels. The viral antigen or reference antigen being labeled with detectors configured for detection of particles of any type or any size, with or without physical modification comprising one of magnetism, with, and without dye labels. These particles will be detected on the surface of the different types of immune cells among circulating white blood cells.

At step 304, cell-bound markers are detected as indicative of immune activation with the viral antigens. The detection of the cell-bound markers is performed using a detection method capable of detecting cell-bound or cell-derived markers from one of flow cytometry, mass cytometry, and single-cell RNA sequencing.

At step 306, an immune cell response is measured using a marker CD154 expressed on stimulated white blood cells of the one or more subjects. The cells which express CD154 are to be measured i.e., the white blood cells comprise T-cells, T-helper cells (CD4), T-cytotoxic cells (CD8) and their memory (CD45RO+) and naïve subsets (CD45RO−), B-cells (CD19 or CD20), natural killer cells (CD16 and/or CD56) and monocytes (CD14). In an embodiment, the immune cell response is measured using markers on cell imaging platforms using one of fluorescence and metal detection.

In an alternative embodiment, the method includes measuring additional markers including but not limited to those expressed on the cell surface which are mentioned in 0013 and 0014. The additional markers include but are not limited to CD38, CD137 or CD69, cytokines such as IFNγ, TNF-α and IL-2, exhaustion markers such as PD-1 and PD-L1, and suppressive markers such as CD39, CD73, FOXP3, TGFb1 and IL-10. The measurements of different cell markers are made with specific antibodies to cell markers which are labeled with detectors such as dyes, fluorescent dyes, and metallic labels for the purpose of detection of particles of any type or any size, with or without physical modification such as magnetism, and with or without dye labels.

At step 308, a risk score is generated using a computing device configured with a multivariate predictive algorithm based on immune cell responses and a plurality of variables associated with a subject. The T-cells, T-helper cells (CD4), T-cytotoxic cells (CD8) and their memory (CD45RO+) and naïve subsets (CD45RO−), B-cells (CD19 or CD20), natural killer cells (CD56 and/or CD16) and monocytes (CD14 or CD16) that express CD154 in response to viral antigens are combined with subject variables including but not limited to age, gender, ethnicity, time from diagnosis of infection with molecular studies, and whether immunosuppressive drugs are used or not, into the multivariate predictive algorithm that generates the risk score.

At step 310, a risk of cell-mediated immunity is determined with cell-mediated immunity to gene therapy based on a magnitude of the risk score. The magnitude of the risk score corresponds to the severity of infection such as respiratory failure requiring mechanical ventilation, and mild or asymptomatic infection. The severity of infection is categorized into one of mild, moderate, and severe based on the magnitude of the risk score. The magnitude of the risk score also determines whether the response to vaccination results in decreased risk of infection or not.

In another embodiment of the present invention, the virus particles comprise Severe Acute Respiratory Syndrome Coronavirus-2, the method for measuring cell-mediated immunity to COVID-19 infection comprises analyzing immune cell responses to the Severe Acute Respiratory Syndrome Coronavirus-2 (SARS-COV-2) antigens in the subject. The risk score is generated utilizing the computing device based on the immune cell responses and the plurality of variables of the subject. The risk of infection, severity of disease, and response to vaccination are predicted against SARS-CoV-2 based on a generated risk score.

The method may be implemented in any suitable hardware, software, firmware, or combination thereof. The order in which the method is described is not intended to be construed as a limitation, and any number of the described method blocks may be combined or otherwise performed in any order to implement the method or an alternate method. Additionally, individual blocks may be deleted from the method without departing from the spirit and scope of the present disclosure described herein. Furthermore, the method may be implemented in any suitable hardware, software, firmware, or a combination thereof, that exists in the related art or that is later developed. The method describes, without limitation, the implementation of the system. A person of skill in the art will understand that method may be modified appropriately for implementation in various manners without departing from the scope and spirit of the disclosure.

In yet another embodiment of the present invention, a kit for measuring cell-mediated immunity to gene therapy is provided. The kit enables rapid measuring of cell-mediated immunity to gene therapy. The kit includes a reference antigen to bind with one or more white blood cells of individuals to be tested for cell-mediated immunity. The reference antigen may comprise virus particles, viral protein, and peptides representing viral proteins.

In an alternative embodiment, the kit comprises the reference antigen labeled with detectors such as dyes, fluorescent dyes, and metallic labels. The viral antigen or the reference antigen is labeled with detectors for a purpose of detection of particles of any type or any size with or without physical modification such as magnetism and with or without dye labels. This detection occurs on the surface of immune cells called antigen-presenting cells. The number of antigen cells that bind these antigens is a measure of the magnitude of cell-mediated immunity.

In an embodiment, the kit or pre-fabricated assay versions or the reference assay system which uses all reagents in the liquid phase including but not limited to additional markers such as CD38, CD137 or CD69, cytokines such as IFNγ, TNF-α and IL-2, exhaustion markers such as PD-1 and PD-L1, and suppressive markers such as CD39, Cd73, FOXP3, TGFb1 and IL-10 to measure the response of reaction between the reference antigen and the one or more white blood cells of individuals to be tested for cell-mediated immunity to AAV gene therapy. The range of marker categories is outlined in 0013 and 0014.

The present invention is explained further in the following specific examples which are only by way of illustration and are not to be construed as limiting the scope of the invention.

### Example 1: Cell-Mediated Immunity to AAV8 (Adeno-Associated Virus 8)

Fresh or frozen peripheral blood leukocytes (PBL) from healthy adults were stimulated with peptide antigen mixtures representing AAV8, a member of an adeno-associated virus family. Mean+/−SEM frequencies of the various blood leukocyte subsets, that expressed the activation marker, CD154, are as follows:

a) T-cells 0.69±0.37, b) B-cells 1.67±0.62, c) natural killer (NK) cells 3.90±1.77, d) NK-T-cells 15.64±8.66, and e) Monocytes 4.66±3.98

This general experimental design can be used to evaluate several marker combinations which include CD154 as one of many activation markers. Further, any type of AAV can be used alone or in combination to elicit the cellular immune response.

In the present invention a) frequencies of SARS-COV-2 reactive CD8 cells that express CD154 in patients with COVID-19 infection, healthy individuals without transplant (healthy) and individuals with transplants (healthy LT), b) performance of the model for predicting COVID-19 infection status in the form of receiver operating-characteristic curve (ROC) along with area under the curve (AUC) of 0.888, and c) a forest plot for presence or absence of immunosuppression with the model variables, are analyzed.

The SARS-COV-2-reactive CD3, CD4 and CD8 cells predicted intubation or no intubation correctly in 86% and 92%, respectively, among the COVID-19 patients. The S2-reactive cells demonstrated differences between unexposed and COVID-19 patients similar to those with S-reactive cells. S1 antigen elicited minimal responses.

The a) frequencies of SARS-COV-2 reactive CD3 cells, CD4 cells and CD8 cells in patients with COVID-19 infection, b) the receiver-operating-characteristic (ROC) curve, and c) forest plot, are also analyzed. Area under the receiver operating characteristic curves was 0.94. The individuals are divided into three groups those requiring intubation, those that did not require intubation but were hospitalized, and those that were asymptomatic. The receiver-operating-characteristic (ROC) curve shows performance of a model that incorporates the three cell types for predicting intubation status. The forest plot shows relative contribution of the three cell types to this optimal model.

(Please provide further experimental data if carried out to compare the efficiency of the method of the present invention or other experimental data or characterization studies performed, if any)

The present invention provides the method for measuring cell-mediated immunity to COVID-19 infection. The method enables measuring the risk of the COVID-19 infection especially to the individuals with transplants. The method enables measuring the severity of the COVID-19 infection such as respiratory failure and requirement of mechanical ventilation. The method also enables measuring response to vaccination against SARS-COV-2, the virus that causes the COVID-19. The method also enables measuring immunity to the virus in healthy individuals that have not been exposed to the virus or have been exposed to the virus, and vulnerable individuals such as those with advanced age, those receiving drugs to modulate, suppress or enhance the immune system, and those with chronic illness. The present invention also provides for a computing device configured to compute the risk score. The present invention also provides the kit or prefabricated reaction tubes/liquid phase reagents for measuring cell-mediated immunity to the COVID-19 infection. The kit enables cost-effective measurement of the cell mediated immunity to the COVID-19 infection.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element.

Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims.

What is claimed is:

1. A method for measuring cell-mediated immunity for viral antigens comprising:
a) mixing white blood cells of one or more subjects to be tested for cell-mediated immunity with viral antigens;
b) detecting a plurality of cell-bound markers indicative of an immune activation with the viral antigens;
c) measuring an immune cell response using a marker CD154 expressed on stimulated white blood cells of the one or more subjects;
d) generating, by a computing device configured with a multivariate predictive module, a risk score based on a measured immune cell response and a plurality of variables associated with a subject; and
e) determining a risk associated with the cell-mediated immunity to gene replacement therapy based on a magnitude of the risk score.

2. The method of claim 1, wherein the viral antigens comprise virus particles, viral proteins, and peptides representing viral proteins.

3. The method of claim 2, wherein the viral antigens comprise adeno-associated viruses (AAV) and transgenes carried by the adeno-associated viruses (AAV) for the gene replacement therapy.

4. The method of claim 1, wherein the viral antigen is labeled with detectors comprising at least one of dyes, fluorescent dyes, and metallic labels.

5. The method of claim 1, wherein the detection of the cell-bound markers is performed using a detection method capable of detecting cell-bound or cell-derived markers from one of flow cytometry, mass cytometry, and single-cell RNA sequencing.

6. The method of claim 1, wherein the white blood cells comprise at least one of T-cells, T-helper cells (CD4), T-cytotoxic cells (CD8) and memory (CD45RO+) and naïve subsets (CD45RO−) of the T-cytotoxic cells, B-cells (CD19 or CD20), natural killer cells (CD16 and CD56) and monocytes (CD14 and CD16).

7. The method of claim 1, further comprising measuring additional markers on a cell surface, wherein the additional markers comprise CD38, CD137, CD69, cytokines (IFNγ, TNF-α, IL-2), exhaustion markers (PD-1, PD-L1), and suppressive markers (CD39, CD73, FOXP3, TGFb1, IL-10).

8. The method of claim 1, wherein the plurality of variables associated with the subject comprises age, gender, ethnicity, time from diagnosis of infection with molecular studies, use of immunosuppressive drugs, and combinations thereof.

9. The method of claim 1, wherein the magnitude of the risk score corresponds to a severity of an infection.

10. The method of claim 1, wherein the severity of infection is categorized into one of mild, moderate, and severe based on the magnitude of the risk score.

11. The method of claim 1, wherein the magnitude of the risk score determines whether the response to vaccination results in decreased risk of infection.

12. The method of claim 1, wherein the virus particles comprise Severe Acute Respiratory Syndrome Coronavirus-2, the method for measuring cell-mediated immunity to COVID-19 infection comprises:
analyzing immune cell responses to the Severe Acute Respiratory Syndrome Coronavirus-2 (SARS-COV-2) antigens in the subject;
generating the risk score utilizing the computing device based on the immune cell responses and the plurality of variables of the subject; and
predicting the risk of infection, severity of disease, and response to vaccination against SARS-COV-2 based on a generated risk score.

13. A computing device for generating a risk score for measuring cell-mediated immunity comprises:
one or more hardware processors configured to run a plurality of modules; and
a memory coupled to the one or more hardware processors, wherein the memory comprises the plurality of modules in form of programmable instructions executable by the one or more hardware processors, wherein the plurality of modules comprises:
a multivariate predictive module configured to generate a risk score based on a measured immune cell response and a plurality of variables associated with a subject;
a determination module configured to determine a risk associated with cell-mediated immunity to gene replacement therapy based on a magnitude of the risk score; and
a result outputting module configured to output, through a display communicatively connected to the one or more hardware processors, for visual presentation of data and results, based on a result of the magnitude of the risk score.

14. A system for measuring cell-mediated immunity for viral antigens, comprising:
a sample processing unit configured to mix white blood cells of one or more subjects with viral antigens;
a detection unit configured to detect cell-bound markers indicative of immune activation with the viral antigens;
an immune cell response measurement unit configured to measure an immune cell response using markers comprising CD154 expressed on stimulated white blood cells; and
a computing device configured for generating a risk score for measuring cell-mediated immunity, comprises:
one or more hardware processors configured to run a plurality of modules; and
a memory coupled to the one or more hardware processors, wherein the memory comprises the plurality of modules in form of programmable instructions executable by the one or more hardware processors, wherein the plurality of modules comprises:
a multivariate predictive module configured to generate a risk score based on a measured immune cell response and a plurality of variables associated with a subject;
a determination module configured to determine a risk associated with cell-mediated immunity to gene replacement therapy based on a magnitude of the risk score; and
a result outputting module configured to output, through a display communicatively connected to the one or more hardware processors, for visual presentation of data and results, based on a result of the magnitude of the risk score.

15. The system of claim 14, wherein the viral antigens comprise virus particles, viral proteins, and peptides representing viral proteins.

16. The system of claim 14, wherein the viral antigens comprise adeno-associated viruses (AAV) and transgenes carried by the adeno-associated viruses (AAV) for the gene replacement therapy.

17. The system of claim 14, wherein the detection of the cell-bound markers is performed using a detection method capable of detecting one of cell-bound and cell-derived markers from one of flow cytometry, mass cytometry, and single-cell RNA sequencing.

18. The system of claim 14, wherein the white blood cells comprise at least one of T-cells, T-helper cells (CD4), T-cytotoxic cells (CD8) and memory (CD45RO+) and naïve subsets (CD45RO−) of the T-cytotoxic cells, B-cells (CD19 or CD20), natural killer cells (CD16 and CD56) and monocytes (CD14).

19. The system of claim 14, further comprising measuring additional markers on a cell surface, wherein the additional markers comprise at least one of CD38, CD137, CD69, cytokines (IFNγ, TNF-α, IL-2), exhaustion markers (PD-1, PD-L1), and suppressive markers (CD39, CD73, FOXP3, TGFb1, IL-10).

20. The system of claim 14, wherein the plurality of variables associated with the subject comprises at least one of age, gender, ethnicity, time from diagnosis of infection with molecular studies, and use of immunosuppressive drugs.
